(19) United States
(12) Patent Application Publication
(10) Pub. No.: US 2025/0079017 A1
(43) Pub. Date: Mar. 6, 2025

(54) INTRAOPERATIVE ON-SPOT ABNORMAL TISSUE DETECTION

(71) Applicant: Duke University, Durham, NC (US)

(72) Inventors: Tanner Zachem, Durham, NC (US); Jacob Sperber, Durham, NC (US); Patrick Codd, Durham, NC (US); C. Rory Goodwin, Durham, NC (US)

(21) Appl. No.: 18/824,144

(22) Filed: Sep. 4, 2024

Related U.S. Application Data
(60) Provisional application No. 63/536,545, filed on Sep. 5, 2023.

(51) Int. Cl. G16H 50/30; G01J 3/02; G01J 3/28

(52) U.S. Cl. CPC G16H 50/30; G01J 3/0208; G01J 3/0218; G01J 3/28; G01J 2003/2856

(57) ABSTRACT

An on-spot abnormal tissue detection system includes a light source, a housing, a light fiber, a collimating lens, one or more beam combining mirrors, one or more focusing optic lenses, one or more achromatic doublet lenses, a spectrometer, a fiber optic cable, and a computing system. The computing system can receive wavelength intensity pairs for spot locations of the target area from the spectrometer, determine a standard deviation and variance measurement of the wavelength intensity pairs for the spot locations, determine whether tissue for the spot locations of the target area is a tissue type of a tumor or normal tissue based on the standard deviation and variance measurement of the wavelength intensity pairs, and send the determination of whether the tissue for the spot locations of the target area is a tissue type of a tumor or normal tissue to a display.

### CROSS-REFERENCE TO RELATED APPLICATIONS

This application claims the benefit of U.S. Provisional Application Ser. No. 63/536,545, filed Sep. 5, 2023.

### BACKGROUND

During surgery to remove abnormal, tumorous, and/or cancerous tissue, surgeons are often required to make judgement calls on the outer periphery of the tissue being removed. In some cases, surgeons must decide whether to remove tissue that may be healthy or cancerous. Furthermore, depending on the region being removed, surgeons may not have a clear view of the tissue itself. Removing healthy tissue can cause undue injury and prolong recovery, while not removing tumorous/pathologic tissue can lead to additional interventions/surgeries placing strain on the healthcare system and/or decline/death of the patient. Current intraoperative methods and systems are available, but are prohibitively expensive, limited to advanced healthcare facilities, and do not yield the desired sensitivity and specificity. Accordingly, there is a need for better intraoperative methods and systems to distinguish healthy tissue from abnormal tissue.

### BRIEF SUMMARY

Systems and methods for intraoperative on-spot abnormal tissue detections are disclosed herein. Advantageously, the systems and methods disclosed herein provide a real-time 3D representation of the target area tissue that identify margins of the abnormal tissue and differentiate the abnormal tissue from normal, healthy tissue. Additionally, the systems and methods disclosed herein can integrate previously gathered information (e.g., pathology report and/or previous scans of the target area tissue) to improve sensitivity and specificity results of the differentiation between abnormal and normal, healthy tissue, and also do not alter the state of the tissue, which allows for post-operative testing of the tissue to confirm the results of the surgery.

In one embodiment, an on-spot abnormal tissue detection system includes a light source, a housing, a light fiber connected to the light source that carries light from the light source into the housing, a collimating lens positioned inside the housing to receive the light carried by the light fiber, one or more beam combining mirrors positioned inside the housing in a light path from the collimating lens, one or more focusing optic lenses positioned inside the housing in the light path from the one or more beam combining mirrors, one or more achromatic doublet lenses positioned inside the housing that focus each wavelength of light reflected from the target area, a spectrometer, a fiber optic cable that carries light from the one or more achromatic doublet lenses to the spectrometer, and a computing system including a processor, storage, and instructions stored on the storage that when executed by the processor, cause the computing system to: receive wavelength intensity pairs for one or more spot locations of the target area from the spectrometer, determine a standard deviation and variance measurement of the wavelength intensity pairs for the one or more spot locations of the target area from the spectrometer, determine whether tissue for the one or more spot locations of the target area is a tissue type of a tumor or normal tissue based on the standard deviation and variance measurement of the wavelength intensity pairs, and send the determination of whether the tissue for the one or more spot locations of the target area is a tissue type of a tumor or normal tissue to a display or output to the surgeon in another manner. The one or more beam combining mirrors reflect light below a particular wavelength and transmit light above the particular wavelength. The one or more focusing optic lenses focus the transmitted light from the one or more beam combining mirrors onto a target area.

In some cases, the instructions stored on the storage that when executed by the processor, further cause the computing system to determine a probability of the determination of whether the tissue for the one or more spot locations of the target area is a tissue type of a tumor or normal tissue based on the standard deviation and variance measurement of the wavelength intensity pairs. In some cases, the instructions stored on the storage that when executed by the processor, further cause the computing system to: receive a previous pathology report for the target area, extract information from the previous pathology report for the target area, and create a feature vector based on the information extracted from the previous pathology report for the target area, and the instructions that determine whether tissue for the one or more spot locations of the target area is a tissue type of a tumor or normal tissue based on the standard deviation and variance measurement of the wavelength intensity pairs comprise instructions that determine whether tissue for the one or more spot locations of the target area is a tissue type of a tumor or normal tissue based on the standard deviation, the variance measurement of the wavelength intensity pairs, and the feature vector based on the information extracted from the previous pathology report for the target area. In some cases, the instructions stored on the storage that when executed by the processor, further cause the computing system to: receive light scattering properties for the one or more spot locations of the target area from the spectrometer, and in response to a determination that the tissue for the one or more spot locations of the target area is a tissue type of a tumor, determine whether the tissue is cancerous, benign, or normal based on the light scattering properties for the one or more spot locations of the target area received from the spectrometer. In some cases, the instructions stored on the storage that when executed by the processor, further cause the computing system to determine a probability of the determination of whether the tissue for the one or more spot locations of the target area is a tissue type of cancerous or benign based on the light scattering properties for the one or more spot locations of the target area received from the spectrometer. In some cases, the instructions stored on the storage that when executed by the processor, further cause the computing system to generate a 3D pathological map using the determination of whether the tissue for the one or more spot locations of the target area is a tissue type of a tumor or normal tissue and a location of each of the one or more spot locations within the target area.

In another embodiment, an on-spot abnormal tissue detection system includes a light source, a housing, a light fiber connected to the light source that carries light from the light source into the housing, a collimating lens positioned inside the housing to receive the light carried by the light fiber, one or more beam combining mirrors positioned inside the housing in a light path from the collimating lens, one or more focusing optic lenses positioned inside the housing in the light path from the one or more beam combining mirrors, one or more achromatic doublet lenses positioned inside the housing that focus each wavelength of light reflected from the target area, a spectrometer, a fiber optic cable that carries light from the one or more achromatic doublet lenses to the spectrometer, and a plurality of navigational fiducials positioned inside the housing that provide a location of the housing to a computing system. The one or more beam combining mirrors reflect light below a particular wavelength and transmit light above the particular wavelength. The one or more focusing optic lenses focus the transmitted light from the one or more beam combining mirrors onto a target area.

In some cases, the on-spot abnormal tissue detection system further includes the computing system. The computing system includes a processor, storage, and instructions stored on the storage that when executed by the processor, cause the computing system to: receive a pre-registered image of the target area, receive, from the light source, timing information, and determine, from the timing information, the location of the housing, and the pre-registered image of the target area, a spot location of the light reflected from the target area.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A and 1B illustrate intraoperative on-spot abnormal tissue detection systems.

FIG. 2A illustrates a flow diagram of a method of determining whether tissue is a tumor or normal tissue.

FIG. 2B illustrates a flow diagram of a method of determining a spot location within a target area.

FIG. 3 illustrates an example architecture of a machine learning model that can be used in an intraoperative on-spot abnormal tissue detection system.

FIGS. 4A-4D illustrate example scan results of tissue using an intraoperative on-spot abnormal tissue detection system.

FIG. 5 illustrates an intraoperative on-spot abnormal tissue detection system being used during surgery on a patient.

FIGS. 6A and 6B illustrate a 3D pathological map that can be generated using an intraoperative on-spot abnormal tissue detection system.

FIG. 7 illustrates a computing system that can be used in an intraoperative on-spot abnormal tissue detection system.

### DETAILED DESCRIPTION

Systems and methods for intraoperative on-spot abnormal tissue detections are disclosed herein. Advantageously, the systems and methods disclosed herein provide a real-time 3D representation of the target area tissue that identify margins of the abnormal tissue and differentiate the abnormal tissue from normal, healthy tissue. Additionally, the systems and methods disclosed herein can integrate previously gathered information (e.g., pathology report and/or previous scans of the target area tissue) to improve sensitivity and specificity results of the differentiation between abnormal and normal, healthy tissue, and also do not alter the state of the tissue, which allows for post-operative testing of the tissue to confirm the results of the surgery.

The Warburg Effect is used by an on-spot abnormal tissue detection system to identify tumor cells, which according to the Warburg Effect, take an usual path versus normal tissue with respect to energy production when light from a light source causes the tissue to fluoresce. For example, the on-spot abnormal tissue detection system can target and/or identify respiration cofactors (e.g., NADH, NAD(P)H, and FAD) that exist in reduced or oxidized form in tissues and fluoresce when light is emitted onto the tissues. Depending on the concentration of these cofactors, the metabolic pathway is different between normal, healthy tissue and tumorous tissue. Therefore, the on-spot abnormal tissue detection system does not measure the concentration and/or ratio of these cofactors, but instead leverage the difference of these cofactors in normal and tumorous tissue to determine a tissue type. In this way, the machine learning model described herein is a “light” computational system that can be leveraged and/or packaged in a small area, allowing the on-spot abnormal tissue detection system to be used inside an operating room, where space is at a premium. In some cases, a machine learning model is not included and other data analysis methods, such as classic statistics, are used in lieu of the machine learning model, which also provides the advantage of being leveraged and/or packaged in a small area, allowing the on-spot subnormal tissue detection system to be used inside an operating room. Another advantage of the on-spot abnormal tissue detection system is that the light from the light source does not alter and/or damage the tissue. Therefore, after surgery, a pathologist can examine the tissue that was removed (e.g., under a microscope) to confirm the results of the on-spot abnormal tissue detection system.

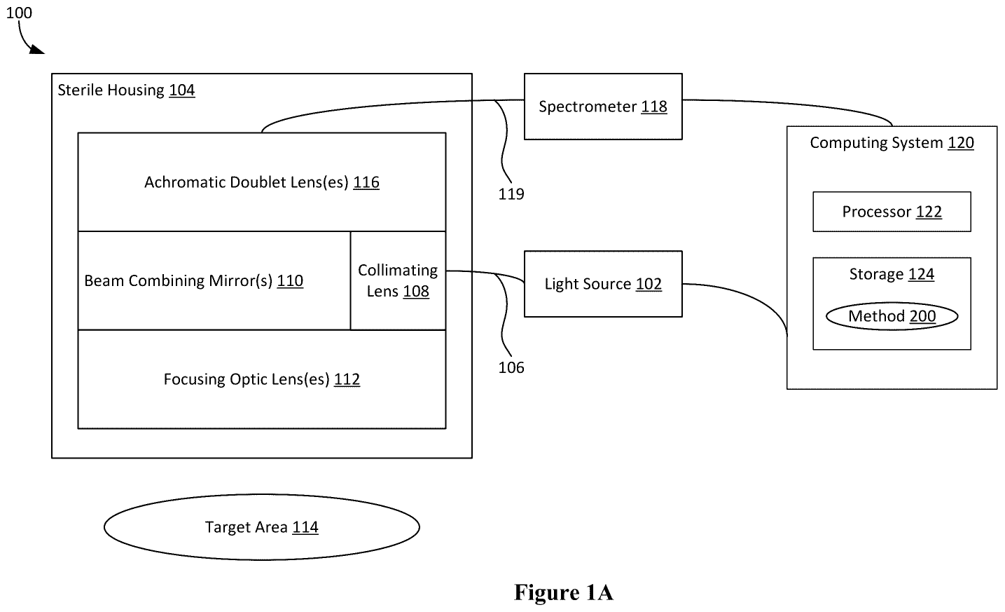

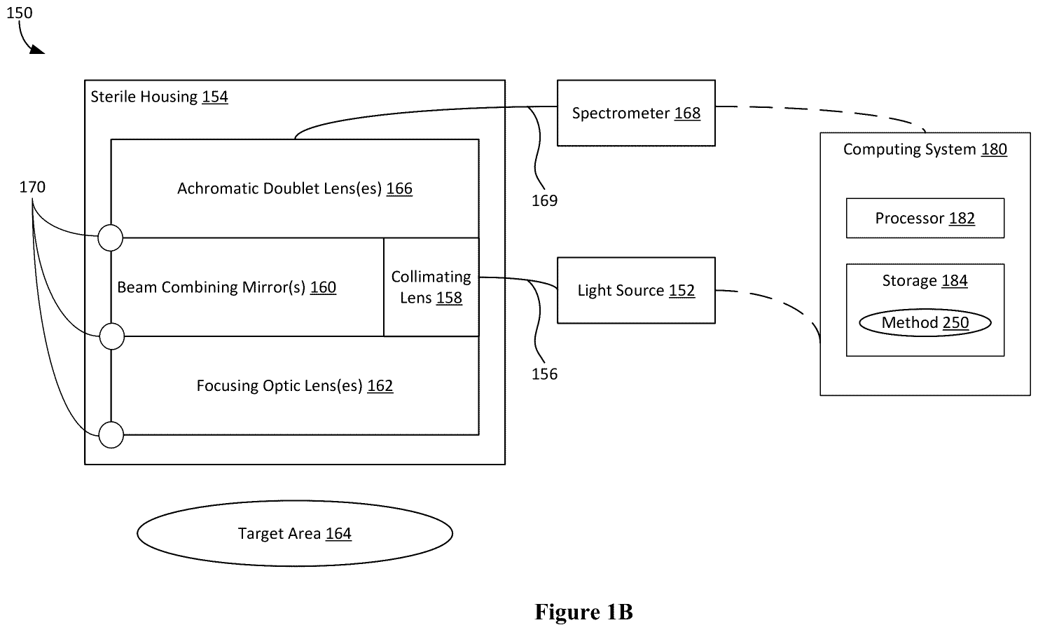

FIGS. 1A and 1B illustrate intraoperative on-spot abnormal tissue detection systems. Referring to FIG. 1A, an intraoperative on-spot abnormal tissue detection system 100 includes a light source 102, a housing 104, a light fiber 106 connected to the light source 102 that carries light from the light source 102 into the housing 104, a collimating lens 108 positioned inside the housing 104 to receive the light carried by the light fiber 106, and one or more beam combining mirrors 110 positioned inside the housing 104 in a light path from the collimating lens 108. The one or more beam combining mirrors 110 reflect light below a particular wavelength and transmit light above the particular wavelength. The intraoperative on-spot abnormal tissue detection system 100 further includes one or more focusing optic lenses 112 positioned inside the housing 104 in the light path from the one or more beam combining mirrors 110. The one or more focusing optic lenses 112 focus the transmitted light from the one or more beam combining mirrors 110 onto a target area 114. The intraoperative on-spot abnormal tissue detection system 100 further includes one or more achromatic doublet lenses 116 positioned inside the housing 104 that focus each wavelength of light reflected from the target area 114, a spectrometer 118, a fiber optic cable 119 that carries light from the one or more achromatic doublet lenses 116 to the spectrometer 118, and a computing system 120. The computing system 120 includes a processor 122, storage 124, and a method 200 stored on the storage 124 as instructions for the computing system 120 that when executed by the processor 122, cause the computing system 120 to perform the method 200.

In some cases, the on-spot abnormal tissue detection system 100 further includes one or more cameras coupled to an outside of the housing 104 and positioned to capture the target area 114 during use of the on-spot abnormal tissue detection system 100. The one or more cameras can be coupled to a surgical display that displays a video feed captured by the one or more cameras. In some cases, computing system 120 includes the surgical display. In some cases, the surgical display is communicatively coupled to the computing system 120. By using the one or more cameras coupled to an outside of the housing 104 and positioned to capture the target area 114 during use of the on-spot abnormal tissue detection system 100, a surgeon can be provided a clear view of the target area 114, which may not be feasible by the surgeon simply looking at the target area 114, for example, because the target area is inside the body of the patient. The housing 104 can be a sterile housing and/or a housing that is sterilized prior to utilization. In some cases, the housing 104 and its associated components are positioned to capture light reflected from the one or more spot locations by the surgeon or other medical professional (e.g., by hand) during surgery. In some cases, the housing 104 and its associated components are positioned to capture light reflected from the one or more spot locations by robotic arms during surgery.

In some cases, the one or more achromatic doublet lenses 116 are positioned above the one or more beam combining mirrors 110 within the housing 104. In some cases, the one or more focusing optic lenses 112 are apochromat lenses. In some cases, the on-spot abnormal tissue detection system 100 further includes one or more noise reduction filters positioned above the one or more beam combining mirrors 110 in the housing 104, the one or more noise reduction filters removing light from the light source that is not reflected from the target area 114. In some cases, the one or more beam combining mirrors 110 include a dichroic mirror. In some cases, the one or more beam combining mirrors 110 utilize a bifurcated fiber bundle. In some cases, the on-spot abnormal tissue detection system 100 further includes a plurality of navigational fiducials positioned inside the housing 104 that provide a location of the housing 104 to the computing system 120.

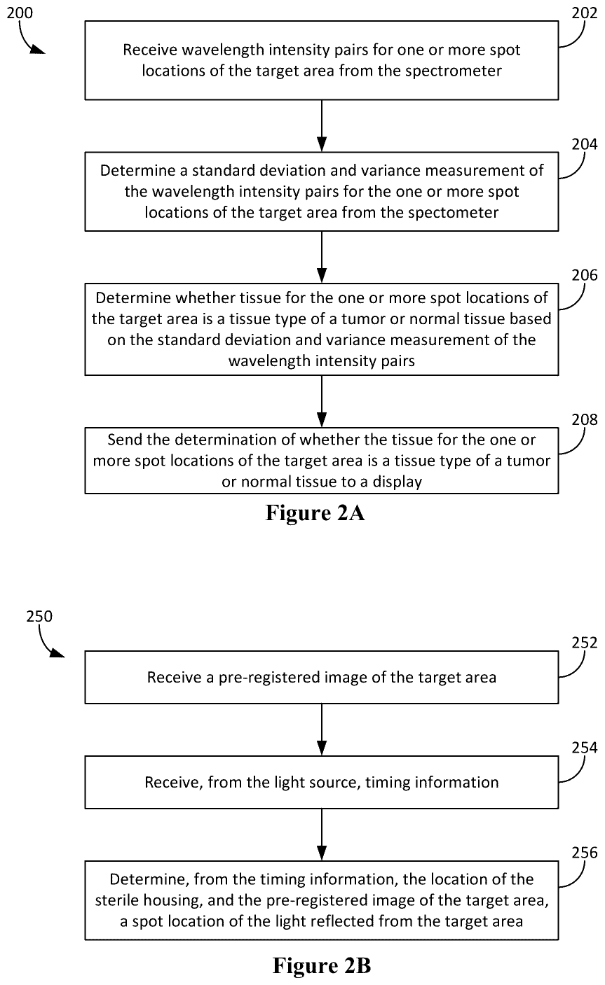

In some cases, the light from the light source 102 includes a wavelength of light ranging from 350 nanometers (nm) to 750 nm. In some cases, the light source 102 is a laser. In some cases, the light source 102 is a low power blue laser. In some cases, the light from the light source 102 will cause the tissue in the target area 114 to fluoresce. Healthy and/or normal tissue fluoresces differently from a tumor, and the computing system 120 can determine, from wavelength intensity pairs from the spectrometer 118, whether tissue is a tissue type of a tumor or normal tissue, as explained in more detail below with respect to FIG. 2A. For example, the light from the light source 102 can excite certain biological markers associated with a tissue type of a tumor or normal tissue, which can be reflected in the wavelength intensity pairs from the spectrometer 118. The biological markers can be, for example, the Warburg Effect. In some cases, the wavelength intensity pairs from the spectrometer 118 can include a wavelength measurement and an intensity measurement.

FIG. 2A illustrates a flow diagram of a method of determining whether tissue is a tumor or normal tissue. Referring to FIGS. 1A and 2A, the method 200, when executed by the processor 122, causes the computing system 120 to: receive (202) wavelength intensity pairs for one or more spot locations of the target area 114 from the spectrometer 118, determine (204) a standard deviation and variance measurement of the wavelength intensity pairs for the one or more spot locations of the target area 114 from the spectrometer 118, determine (206) whether tissue for the one or more spot locations of the target area 114 is a tissue type of a tumor or normal tissue based on the standard deviation and variance measurement of the wavelength intensity pairs, and send (208) the determination of whether the tissue for the one or more spot locations of the target area 114 is a tissue type of a tumor or normal tissue to a display. In some cases, the on-spot abnormal tissue detection system 100 further includes the display. In some cases, the display includes and/or is the surgical display. In some cases, each of the one or more spot locations of the target area 114 is scanned for less than 0.25 seconds and the surface area of each of the one or more spot locations of the target area 114 that is scanned is 1 millimeter or less in diameter.

In some cases, the method 200, when executed by the processor 122, further causes the computing system 120 to determine a probability of the determination of whether the tissue for the one or more spot locations of the target area 114 is a tissue type of a tumor or normal tissue based on the standard deviation and variance measurement of the wavelength intensity pairs. In some cases, the method 200, when executed by the processor 122, further causes the computing system 120 to: receive a previous pathology report for the target area 114, extract information from the previous pathology report for the target area 114, and create a feature vector based on the information extracted from the previous pathology report for the target area 114, and the instructions that determine (206) whether tissue for the one or more spot locations of the target area 114 is a tissue type of a tumor or normal tissue based on the standard deviation and variance measurement of the wavelength intensity pairs include instructions that determine whether tissue for the one or more spot locations of the target area 114 is a tissue type of a tumor or normal tissue based on the standard deviation, the variance measurement of the wavelength intensity pairs, and the feature vector based on the information extracted from the previous pathology report for the target area 114.

In some cases, the method 200, when executed by the processor 122, further causes the computing system 120 to: receive light scattering properties for the one or more spot locations of the target area 114 from the spectrometer 118, and in response to a determination that the tissue for the one or more spot locations of the target area 114 is a tissue type of a tumor, determine whether the tissue is cancerous or benign based on the light scattering properties for the one or more spot locations of the target area 114 received from the spectrometer 118. In some cases, the method 200, when executed by the processor 122, further causes the computing system 120 to determine a probability of the determination of whether the tissue for the one or more spot locations of the target area is a tissue type of cancerous or benign based on the light scattering properties for the one or more spot locations of the target area 114 received from the spectrometer 118. In some cases, the light scattering properties are an inherent tissue property that are measured, for example, indirectly by the spectrometer 118. In some cases, the determination (206) of whether tissue for the one or more spot locations of the target area 114 is a tissue type of a tumor or normal tissue based on the standard deviation and variance measurement of the wavelength intensity pairs further includes determining whether the tissue for the one or more spot locations of the target area 114 is a tissue type of a tumor or normal tissue based on the standard deviation, the variance measurement of the wavelength intensity pairs, and the light scattering properties. In some cases, other types of tissue classification, such as margin, can be identified using methods similar to those described above for determining tumor or normal and/or cancerous or benign. For example, wavelength intensity pairs and/or light scattering properties for the one or more spot locations of the target area 114 can be used to determine a tissue type of normal (as well as, in some cases, the type of normal tissue), tumor (as well as, in some cases, the grade of the tumor), cancerous (as well as, in some cases, the type of cancer), benign, and/or margin tissue. In some cases, the tissue classification of margin includes tissue that is a mix of normal and abnormal (e.g., tumor) for that corresponding spot location.

In some cases, the method 200, when executed by the processor 122, further causes the computing system 120 to generate a 3D pathological map using the determination of whether the tissue for the one or more spot locations of the target area 114 is a tissue type of a tumor or normal tissue and a location of each of the one or more spot locations within the target area 114. In some cases, the location of each of the one or more spot locations within the target area 114 is determined, at least in part, from a plurality of navigational fiducials positioned inside the housing 104 that provide a location of the housing 104 to the computing system 120. In some cases, the location provided by the plurality of fiducials inside the housing 104 includes a collection of single point collections, a swept series of point collections, a rasterized series of point collections, and/or any other means of collecting location data from the plurality of fiducials inside the housing 104 that can be used to determine the location of each of the one or more spot locations within the target area 114.

In some cases, timing information can also be included in the determination of the location of each of the one or more spot locations within the target area 114. For example, timing information can include timing of when the light from the light source 102 is sent and/or received, and the location of the housing 104 can include the location of the housing 104 provided by the plurality of navigational fiducials. In some cases, distance information can also be included in the determination of the location of each of the one or more spot locations within the target area 114. For example, distance information can include distance of the light from the target area 114, and the location of the housing 104 can include the location of the housing 104 provided by the plurality of navigational fiducials.

Referring to FIG. 1B, an intraoperative on-spot abnormal tissue detection system 150 includes a light source 152, a housing 154, a light fiber 156 connected to the light source 152 that carries light from the light source 152 into the housing 154, a collimating lens 158 positioned inside the housing 154 to receive the light carried by the light fiber 156, and one or more beam combining mirrors 160 positioned inside the housing 154 in a light path from the collimating lens 158. The one or more beam combining mirrors 160 reflect light below a particular wavelength and transmit light above the particular wavelength. The intraoperative on-spot abnormal tissue detection system 150 further includes one or more focusing optic lenses 162 positioned inside the housing 154 in the light path from the one or more beam combining mirrors 160. The one or more focusing optic lenses 162 focus the transmitted light from the one or more beam combining mirrors 160 onto a target area 164. The intraoperative on-spot abnormal tissue detection system 150 further includes one or more achromatic doublet lenses 166 positioned inside the housing 154 that focus each wavelength of light reflected from the target area 164, a spectrometer 168, a fiber optic cable 169 that carries light from the one or more achromatic doublet lenses 116 to the spectrometer 118. The intraoperative on-spot abnormal tissue detection system 150 further includes a plurality of navigational fiducials 170 positioned inside the housing 154 that provide a location of the housing 154 to a computing system 180. The computing system 180 includes a processor 182, storage 184, and a method 250 stored on the storage 184 as instructions for the computing system 180 that when executed by the processor 182, cause the computing system 180 to perform the method 250. In some cases, the intraoperative on-spot abnormal tissue detection system 150 further includes the computing system 180. In some cases, the intraoperative on-spot abnormal tissue detection system 150 further includes any of the features described above with respect to FIGS. 1A and/or 1B.

FIG. 2B illustrates a flow diagram of a method of determining a spot location within a target area. Referring to FIGS. 1B and 2B, the method 250, when executed by the processor 182, causes the computing system 180 to: receive (252) a pre-registered image of the target area 164, receive (254), from the light source 152, timing information, and determine (256), from the timing information, the location of the housing 154, and the pre-registered image of the target area 164, a spot location of the light reflected from the target area 164. For example, the timing information can include timing of when the light from the light source 152 is sent and/or received, and the location of the housing 154 can include the location of the housing 154 provided by the plurality of fiducials 170. In some cases, distance information can also be included in the determination (256) of the spot location of the light reflected from the target area 164. In some cases, the pre-registered image of the target area 164 is generated from another imaging modality, such as MRI, CT, PET, Ultrasound, and/or microscopy.

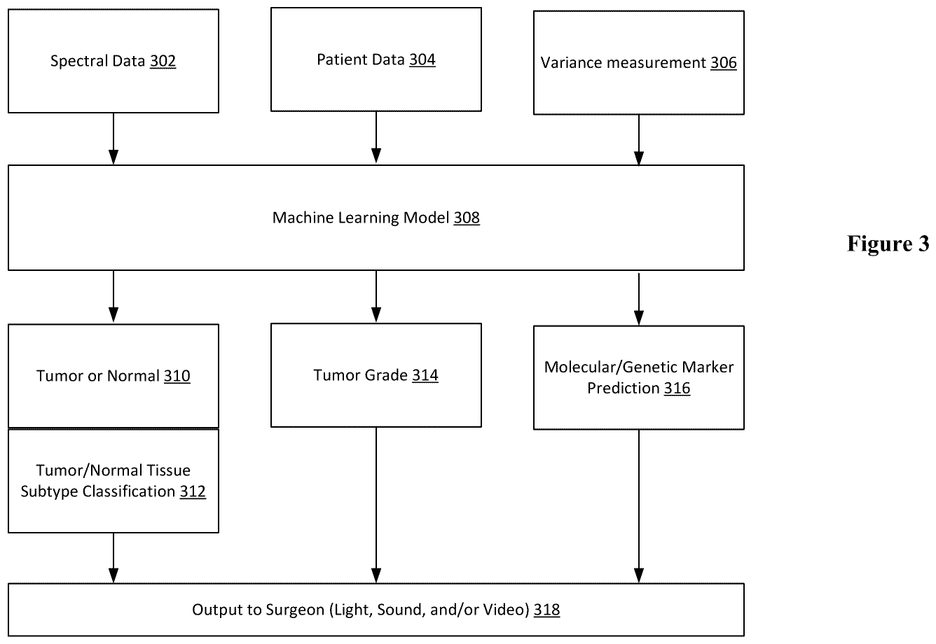

FIG. 3 illustrates an example architecture of a machine learning model that can be used in an intraoperative on-spot abnormal tissue detection system. Referring to FIG. 3, spectral data 302, patient data 304, and variance measurements 306 from each of the one or more spot locations of the target area can be inputted into and/or received by a machine learning model 308. In some cases, the spectral data includes wavelength intensity pairs for the one or more spot locations of the target area from the spectrometer, light scattering properties for the one or more spot locations of the target area from the spectrometer, timing information, distance information, and/or location information. In some cases, the patient data 304 can include a pre-registered image of the target area and/or a previous pathology report. In some cases, the variance measurements 306 can include a standard deviation and/or variance measurement of the wavelength intensity pairs for the one or more spot locations of the target area from the spectrometer. In some cases, the machine learning model 308 can determine a standard deviation and/or variance measurement of the wavelength intensity pairs for the one or more spot locations of the target area from the spectrometer from the wavelength intensity pairs themselves. In some cases, the machine learning model 308 is a deep neural network and/or a support vector machine.

The machine learning model 308 can output, based on the spectral data 302, the patient data 304, and the variance measurements 306 from each of the one or more spot locations of the target area, a determination of whether tissue from each of the one or more spot locations of the target area is a tissue type of a tumor or normal tissue 310 and a type of tumor 312, such as cancerous or benign, and/or what type of normal tissue 312, such as muscle, dura, bone, and other types of normal, healthy tissue. In some cases, the machine learning model 308 can further output a determination of a tumor grade 314, and a molecular and/or genetic marker prediction 316. The determination of the whether tissue from each of the one or more spot locations of the target area is a tissue type of a tumor or normal tissue 310, the type of tumor and/or normal tissue 312, the tumor grade 314, and/or the molecular and/or genetic marker prediction 316 can be output 318 to a surgeon with light, sound, and/or video. For example, the output 318 to the surgeon can be to a visual output to a display and/or surgical display such as video, text, shading, highlighting, and/or coloring of features and/or locations, including spot locations, in the target area, a light visible on the exterior the housing such as green for normal tissue and red for a tumor, and/or sound corresponding to one or more of the determinations 312, 314, and 316.

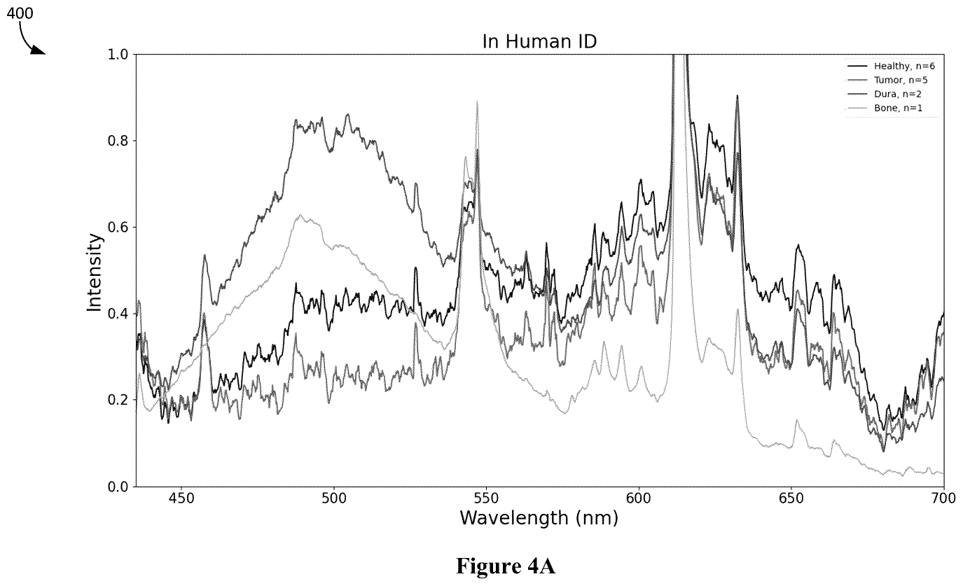

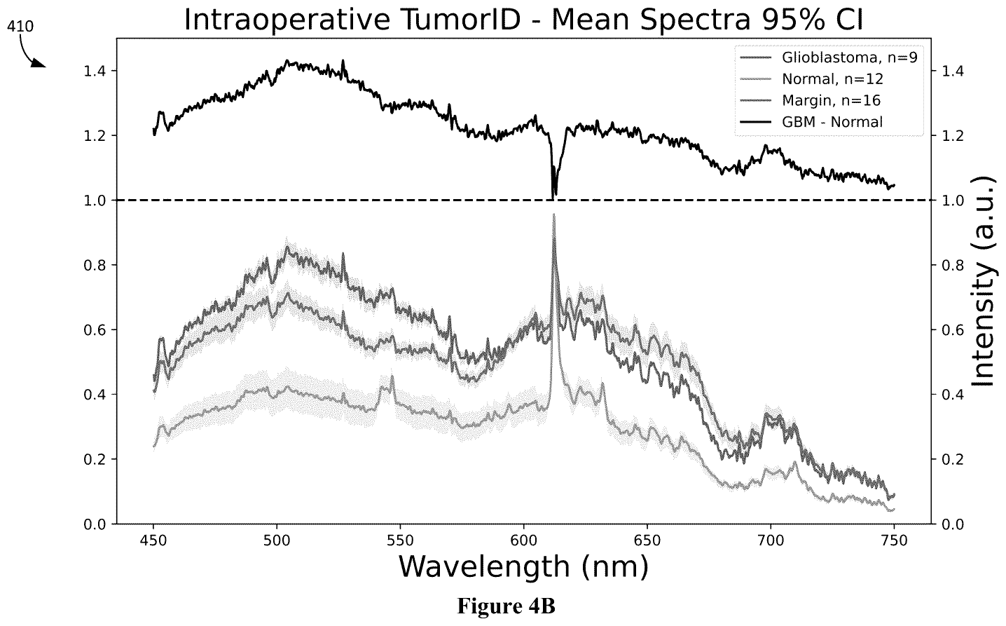

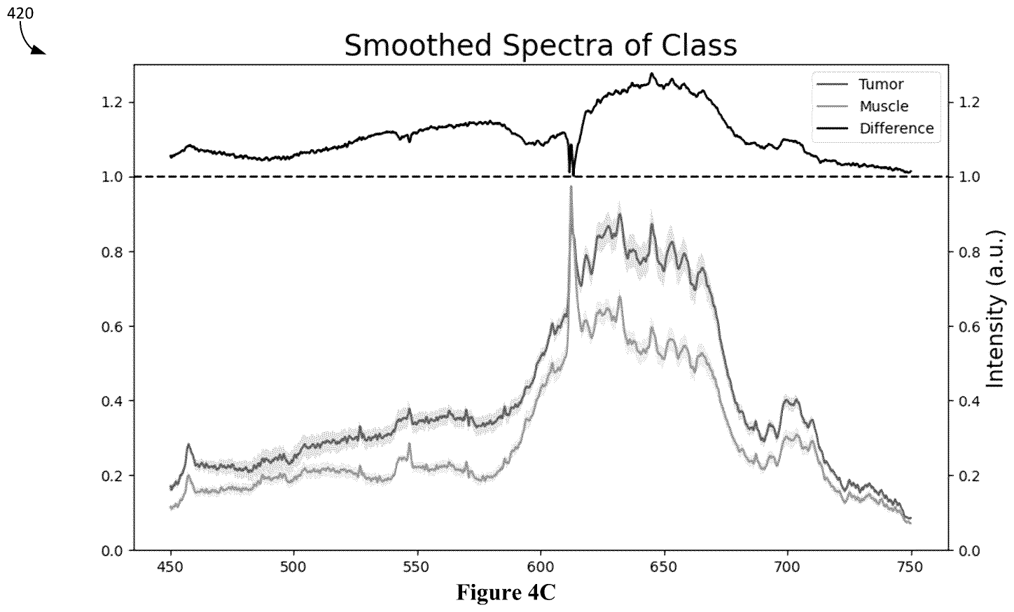

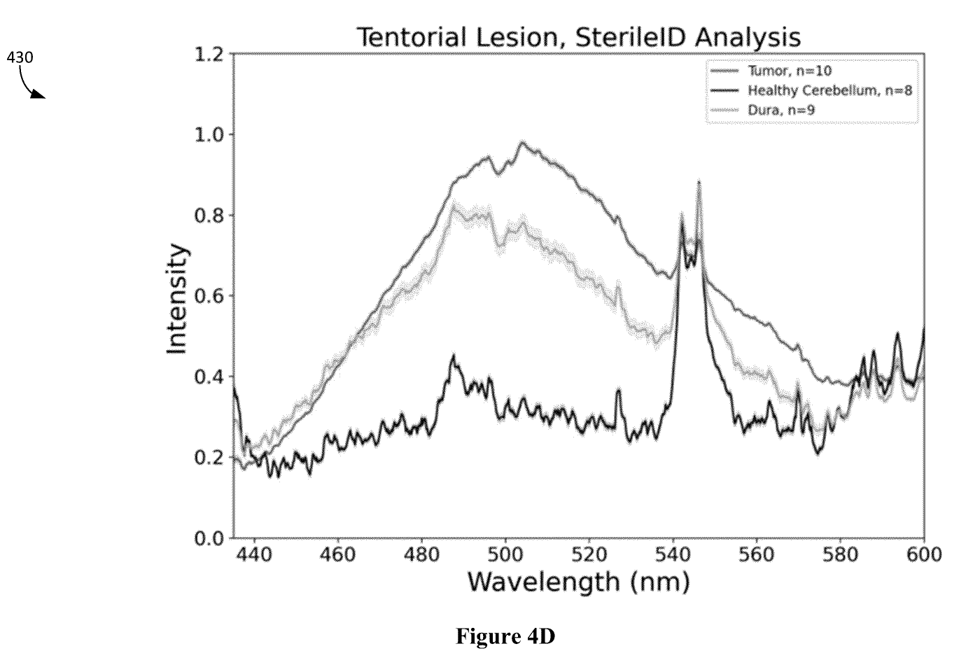

FIGS. 4A-4D illustrate example scan results of tissue using an intraoperative on-spot abnormal tissue detection system. Referring to FIG. 4A, a wavelength versus intensity graphical representation 400 is measured for healthy tissue, tumor tissue, dura tissue, and bone. As can be seen, these different tissue types are distinguishable by their intensities at certain wavelengths.

Referring to FIG. 4B, a wavelength versus intensity graphical representation 410 is measured for Glioblastoma tissue, normal tissue, margin tissue, and a difference measurement between Glioblastoma tissue and normal tissue (GBM-Normal). As can be seen, these different tissue types are distinguishable by their intensities at certain wavelengths with a 95% confidence interval.

Referring to FIG. 4C, a wavelength versus intensity graphical representation 420 is measured for tumor tissue and muscle tissue, and a difference measurement between tumor tissue and normal tissue. As can be seen, these different tissue types are distinguishable by their intensities at certain wavelengths.

Referring to FIG. 4D, a wavelength versus intensity graphical representation 430 is measured for tumor tissue, healthy Cerebellum tissue, and dura tissue. As can be seen, these different tissue types are distinguishable by their intensities at certain wavelengths.

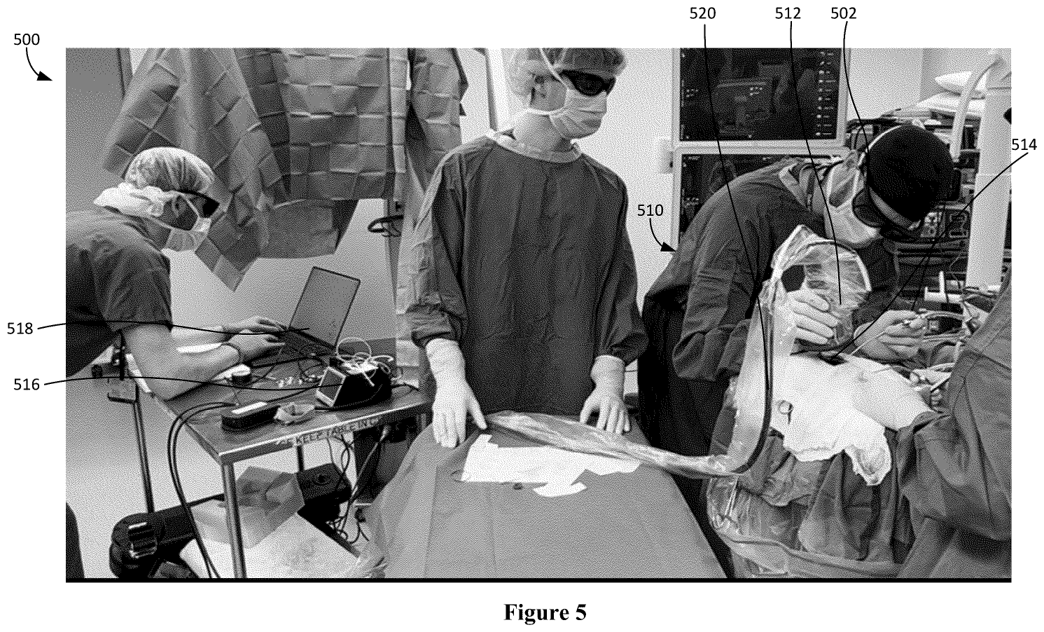

FIG. 5 illustrates an intraoperative on-spot abnormal tissue detection system being used during surgery on a patient. Referring to FIG. 5, an operating room 500 is illustrated. A surgeon 502 uses an intraoperative on-spot abnormal tissue detection system 510 having a housing 512 (including the components described above contained within the housing 512 to identify tissue types of a target area 514 during a surgical procedure. The housing 512 is communicatively coupled to a light source 516 and a computing system 518 by fiber cables 520. As can be seen, the surgeon 502 can use the on-spot abnormal tissue detection system 510 to determine whether to remove tissue during a surgical procedure on a patient.

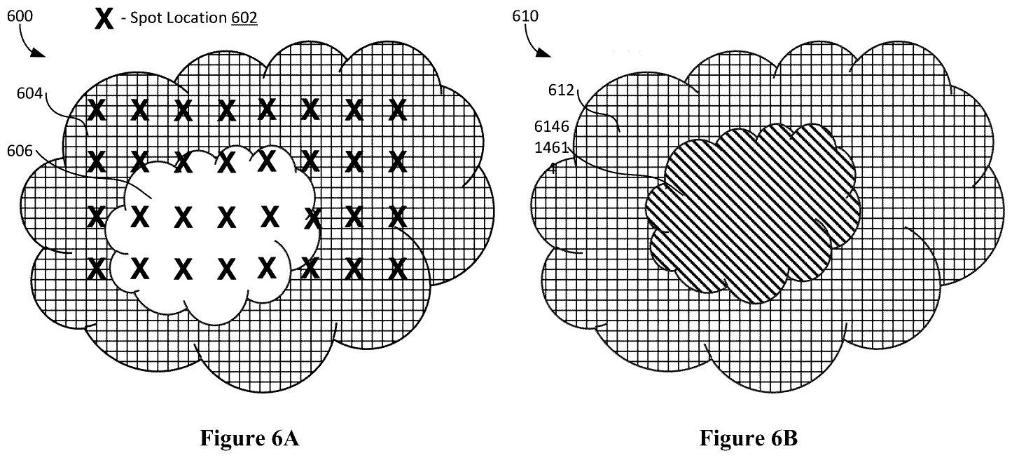

FIGS. 6A and 6B illustrate a 3D pathological map that can be generated using an intraoperative on-spot abnormal tissue detection system. Referring to FIG. 6A, a target area 600 having normal tissue 604 and tumorous tissue 606 is analyzed by an on-spot abnormal tissue detection system at a plurality of spot locations 602. In some cases, the machine learning model can use a pre-registered image of the target area 600 and the plurality of spot locations 602 measured by the on-spot abnormal tissue detection system to generate a 3D pathological map 610. For example, the 3D pathological map 610 can be generated by using the determination of whether the tissue for the plurality of spot locations 602 of the target area 600 is a tissue type of a tumor or normal tissue and a location of each of the spot locations within the target area 600, for example, as measured by the on-spot abnormal tissue detection system and correlated to a pre-registered image of the target area 600 from another imaging modality, such as MRI, CT, PET, Ultrasound, and/or microscopy. The 3D pathological map 610 can include tissue identified as normal 612 and tissue identified as tumor 614.

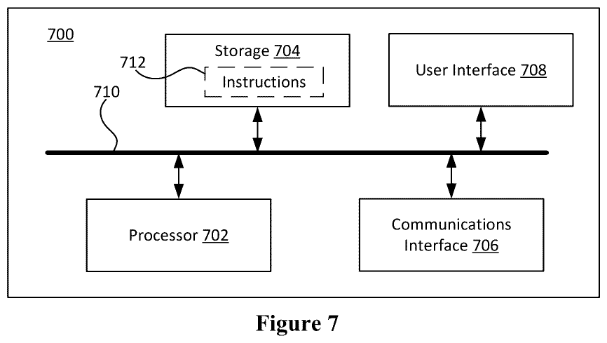

FIG. 7 illustrates a computing system that can be used in an intraoperative on-spot abnormal tissue detection system. Referring to FIG. 7, a computing system 700 can include a processor 702, storage 704, a communications interface 706, and a user interface 708 coupled, for example, via a system bus 710. Processor 702 can include one or more of any suitable processing devices (“processors”), such as a microprocessor, central processing unit (CPU), graphics processing unit (GPU), field programmable gate arrays (FPGAs), application-specific integrated circuits (ASICs), logic circuits, state machines, application-specific standard products (ASSPs), system-on-a-chip systems (SOCs), complex programmable logic devices (CPLDs), etc. Storage 704 can include any suitable storage media that can store instructions 712 and data for performing on-spot abnormal tissue detection, such as for example, method 200 and/or method 250. Suitable storage media for storage 704 includes random access memory, read only memory, magnetic disks, optical disks, CDs, DVDs, flash memory, magnetic cassettes, magnetic tape, magnetic disk storage or other magnetic storage devices, or any other suitable storage media. As used herein, “storage,” “memory storage,” and “storage media” do not consist of transitory, propagating waves. Instead, these terms refer to non-transitory media.

Communications interface 706 can include wired or wireless interfaces for communicating with a light source and/or a spectrometer, such as described with respect to FIGS. 1A and 1B, as well as interfaces for communicating with the “outside world” (e.g., external networks).

User interface 708 can include output device interfaces such as a display and/or surgical display, a light, and/or sound on which the output from applications including the machine learning model running on the computing system 700 can be outputted, as well as suitable input device interfaces for receiving user input (e.g., mouse, keyboard, microphone).

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. An on-spot abnormal tissue detection system comprising:
a light source;
a housing;
a light fiber connected to the light source that carries light from the light source into the housing;
a collimating lens positioned inside the housing to receive the light carried by the light fiber;
one or more beam combining mirrors positioned inside the housing in a light path from the collimating lens, the one or more beam combining mirrors reflecting light below a particular wavelength and transmitting light above the particular wavelength;
one or more focusing optic lenses positioned inside the housing in the light path from the one or more beam combining mirrors, the one or more focusing optic lenses focusing the transmitted light from the one or more beam combining mirrors onto a target area;
one or more achromatic doublet lenses positioned inside the housing that focus each wavelength of light reflected from the target area;
a spectrometer;
a fiber optic cable that carries light from the one or more achromatic doublet lenses to the spectrometer; and
a computing system comprising a processor, storage, and instructions stored on the storage that when executed by the processor, cause the computing system to:
receive wavelength intensity pairs for one or more spot locations of the target area from the spectrometer;
determine a standard deviation and variance measurement of the wavelength intensity pairs for the one or more spot locations of the target area from the spectrometer;
determine whether tissue for the one or more spot locations of the target area is a tissue type of a tumor or normal tissue based on the standard deviation and variance measurement of the wavelength intensity pairs; and
send the determination of whether the tissue for the one or more spot locations of the target area is a tissue type of a tumor or normal tissue to a display.

2. The on-spot abnormal tissue detection system of claim 1, wherein the instructions stored on the storage that when executed by the processor, further cause the computing system to determine a probability of the determination of whether the tissue for the one or more spot locations of the target area is a tissue type of a tumor or normal tissue based on the standard deviation and variance measurement of the wavelength intensity pairs.

3. The on-spot abnormal tissue detection system of claim 1, wherein the instructions stored on the storage that when executed by the processor, further cause the computing system to:
receive a previous pathology report for the target area;
extract information from the previous pathology report for the target area; and
create a feature vector based on the information extracted from the previous pathology report for the target area;
wherein the instructions that determine whether tissue for the one or more spot locations of the target area is a tissue type of a tumor or normal tissue based on the standard deviation and variance measurement of the wavelength intensity pairs comprise instructions that determine whether tissue for the one or more spot locations of the target area is a tissue type of a tumor or normal tissue based on the standard deviation, the variance measurement of the wavelength intensity pairs, and the feature vector based on the information extracted from the previous pathology report for the target area.

4. The on-spot abnormal tissue detection system of claim 1, wherein the instructions stored on the storage that when executed by the processor, further cause the computing system to:
receive light scattering properties for the one or more spot locations of the target area from the spectrometer; and
in response to a determination that the tissue for the one or more spot locations of the target area is a tissue type of a tumor, determine whether the tissue is cancerous or benign based on the light scattering properties for the one or more spot locations of the target area received from the spectrometer.

5. The on-spot abnormal tissue detection system of claim 4, wherein the instructions stored on the storage that when executed by the processor, further cause the computing system to determine a probability of the determination of whether the tissue for the one or more spot locations of the target area is a tissue type of cancerous, benign, or normal based on the light scattering properties for the one or more spot locations of the target area received from the spectrometer.

6. The on-spot abnormal tissue detection system of claim 1, wherein the instructions stored on the storage that when executed by the processor, further cause the computing system to generate a 3D pathological map using the determination of whether the tissue for the one or more spot locations of the target area is a tissue type of a tumor or normal tissue and a location of each of the one or more spot locations within the target area.

7. The on-spot abnormal tissue detection system of claim 1, further comprising one or more cameras coupled to an outside of the housing and positioned to capture the target area during use of the on-spot abnormal tissue detection system, the one or more cameras coupled to a surgical display that displays a video feed captured by the one or more cameras.

8. The on-spot abnormal tissue detection system of claim 1, wherein the one or more achromatic doublet lenses are further positioned above the one or more beam combining mirrors within the housing.

9. The on-spot abnormal tissue detection system of claim 1, wherein the one or more focusing optic lenses are apochromat lenses.

10. The on-spot abnormal tissue detection system of claim 1, further comprising one or more noise reduction filters positioned above the one or more beam combining mirrors in the housing, the one or more noise reduction filters removing light from the light source that is not reflected from the target area.

11. The on-spot abnormal tissue detection system of claim 1, wherein the one or more beam combining mirrors comprise a dichroic mirror.

12. The on-spot abnormal tissue detection system of claim 1, further comprising a plurality of navigational fiducials positioned inside the housing that provide a location of the housing to the computing system.

13. The on-spot abnormal tissue detection system of claim 12, wherein the light source is communicatively coupled to the computing system, wherein the instructions stored on the storage that when executed by the processor, further cause the computing system to:
receive a pre-registered image of the target area;
receive, from the light source, timing information; and
determine, from the timing information, the location of the housing, and the pre-registered image of the target area, a spot location of the light reflected from the target area.

14. The on-spot abnormal tissue detection system of claim 1, wherein the light from the light source comprises a wavelength of light ranging from 350 nm to 700 nm.

15. An on-spot abnormal tissue detection system comprising:
a light source;
a housing;
a light fiber connected to the light source that carries light from the light source into the housing;
a collimating lens positioned inside the housing to receive the light carried by the light fiber;
one or more beam combining mirrors positioned inside the housing in a light path from the collimating lens, the one or more beam combining mirrors reflecting light below a particular wavelength and transmitting light above the particular wavelength;
one or more focusing optic lenses positioned inside the housing in the light path from the one or more beam combining mirrors, the one or more focusing optic lenses focusing the transmitted light from the one or more beam combining mirrors onto a target area;
one or more achromatic doublet lenses positioned inside the housing that focus each wavelength of light reflected from the target area;
a spectrometer;
a fiber optic cable that carries light from the one or more achromatic doublet lenses to the spectrometer; and
a plurality of navigational fiducials positioned inside the housing that provide a location of the housing to a computing system.

16. The on-spot abnormal tissue detection system of claim 15, further comprising the computing system, wherein the computing system comprises a processor, storage, and instructions stored on the storage that when executed by the processor, cause the computing system to:
receive a pre-registered image of the target area;
receive, from the light source, timing information; and
determine, from the timing information, the location of the housing, and the pre-registered image of the target area, a spot location of the light reflected from the target area.

17. The on-spot abnormal tissue detection system of claim 15, further comprising one or more cameras coupled to an outside of the housing and positioned to capture the target area during use of the on-spot abnormal tissue detection system, the one or more cameras coupled to a surgical display that displays a video feed captured by the one or more cameras.

18. The on-spot abnormal tissue detection system of claim 15, further comprising the computing system, wherein the computing system comprises a processor, storage, and instructions stored on the storage that when executed by the processor, cause the computing system to:
receive wavelength intensity pairs for one or more spot locations of the target area from the spectrometer;
determine a standard deviation and variance measurement of the wavelength intensity pairs from the spectrometer;
determine whether tissue of the target area is a tumor or normal tissue based on the standard deviation and variance measurement of the wavelength intensity pairs;
determine a probability of the determination of whether the tissue is a tumor or normal tissue based on the standard deviation and variance measurement of the wavelength intensity pairs; and
send the determination of whether the tissue of the target area is a tumor or normal tissue and the determination of the probability of the determination of whether the tissue is a tumor or normal tissue to a display.

19. The on-spot abnormal tissue detection system of claim 18, wherein the instructions stored on the storage that when executed by the processor, further cause the computing system to:
receive a previous pathology report for the target area;
extract information from the previous pathology report for the target area; and
create a feature vector based on the information extracted from the previous pathology report for the target area;
wherein the instructions that determine whether tissue of the target area is a tumor or normal tissue based on the standard deviation and variance measurement of the wavelength intensity pairs comprise instructions that determine whether tissue of the target area is a tumor or normal tissue based on the standard deviation, the variance measurement of the wavelength intensity pairs, and the feature vector based on the information extracted from the previous pathology report for the target area.

20. The on-spot abnormal tissue detection system of claim 18, wherein the instructions stored on the storage that when executed by the processor, further cause the computing system to:
receive light scattering properties from the spectrometer for the one or more spot locations of the target area; and
in response to a determination that the tissue is a tumor, determine whether the tissue is cancerous, benign, or normal based on the light scattering properties for the one or more spot locations of the target area received from the spectrometer.
